(19) United States
(12) Patent Application Publication
(10) Pub. No.: US 2012/0323085 A1
(43) Pub. Date: Dec. 20, 2012

(54) INFORMATION PROCESSING DEVICE, INFORMATION PROCESSING METHOD, AND INFORMATION PROCESSING PROGRAM

(76) Inventor: Haruto TAKEDA, Tokyo (JP)

(21) Appl. No.: 13/599,554

(22) Filed: Aug. 30, 2012

(30) Foreign Application Priority Data
Jun. 9, 2011 (JP) 2011-193527

(51) Int. Cl. A61B 5/00; G06F 17/30

(52) U.S. Cl. 600/300; 707/736; 707/E17.005

(57) ABSTRACT

There is provided an information processing device including an analysis unit configured to analyze a hypnogram representing a time-series change of a sleep state of a subject and to calculate a sleep parameter representing a feature of sleep of the subject, and an estimation unit configured to estimate an evaluation on sleep of the subject based on a database, in which a diagnosis result of the hypnogram as sample data is registered, and the calculated sleep parameter.

### BACKGROUND

The present disclosure relates to an information processing device, an information processing method and an information processing program, and more particularly, to an information processing device, an information processing method and an information processing program, capable of evaluating the quality of sleep based on a bio-signal detected from a subject during sleep.

In related art, when examining a patient suffering from a sleep disorder or the like in a hospital or the like, a doctor has measured brain waves, ocular potential, myogenic potential and the like from the patient during sleep and has used a hypnogram representing a time-series transition of sleep states, which is created based on the measured brain waves and the like. Such a specialist as a doctor may read a factor causing exacerbation of the quality of sleep, such as an arousal response that does not remain in consciousness, from the hypnogram without making a diagnosis of disease.

On the other hand, there have been a sensor for measuring a heartbeat, a pulse wave or the like, and an analysis device with an application program for analyzing a sleep state based on the measurement result, which have been introduced to general households. (e.g., see Japanese Patent Application Publication No. 2011-115188).

### SUMMARY

For a general household, it will be convenient to obtain the same as a diagnosis result of a doctor regarding the quality of sleep based on a measurement result of a bio-signal, such as a pulse during sleep. Further, the general public suffering from no severe symptom, such as sleep disorder, may want to know how his or her sleep is evaluated by such a specialist as a doctor. However, the above-mentioned analysis device merely estimates the deepness of sleep but does not provide a user with evaluation of such a specialist as a doctor on the quality of sleep.

In view of the foregoing situation, the present disclosure is directed to a technology for evaluating the quality of sleep based on a measurement result of a bio-signal during sleep.

According to an embodiment of the present disclosure, there is provided an information processing device, including an analysis unit configured to analyze a hypnogram representing a time-series change of a sleep state of a subject and to calculate a sleep parameter representing a feature of sleep of the subject, and an estimation unit configured to estimate an evaluation on sleep of the subject based on a database, in which a diagnosis result of the hypnogram as sample data is registered, and the calculated sleep parameter.

According to an embodiment of the present disclosure, the information processing device may further include a presentation unit configured to present the estimated evaluation on the sleep of the subject.

According to an embodiment of the present disclosure, the information processing device may further include a creation unit configured to create an evaluation statement based on the estimated evaluation on the sleep of the subject. The presentation unit may further present the evaluation statement.

The database may register one or more results obtained by diagnosis performed by one or more medical specialists with respect to the hypnogram as sample data.

The estimation unit may include an input unit to input a personal opinion on sleep of the subject and is configured to specify, out of the one or more medical specialists having registered diagnosis results in the database, a medical specialist, an evaluation value estimated based on a result obtained by diagnosis performed by the specified medical specialist being similar to the input personal opinion of the subject.

The estimation unit may be configured to estimate an evaluation on sleep of the subject based on the calculated sleep parameter and the result obtained by diagnosis performed by the specified medical specialist among diagnosis results registered in the database.

The analysis unit may be configured to calculate, as the sleep parameter, at least one of sleep onset, TST/SPT, SWS(% SPT), WASO(% SPT), transition frequency between stages S1 and S2, REM latency, or REM(% SPT).

According to an embodiment of the present disclosure, the information processing device may further include an acquisition unit configured to acquire a bio-signal measured from a subject during sleep, and a generation unit configured to generate the hypnogram based on the acquired bio-signal.

According to an embodiment of the present disclosure, the information processing device may further include a bio-sensor configured to measure the bio-signal from a subject during sleep.

According to an embodiment of the present disclosure, there is provided an information processing method of an information processing device, the information processing method performed by the information processing device, including analyzing a hypnogram representing a time-series change of a sleep state of a subject and calculating a sleep parameter representing a feature of sleep of the subject, and estimating an evaluation on sleep of the subject based on a database, in which a diagnosis result of the hypnogram as sample data is registered, and the calculated sleep parameter.

According to an embodiment of the present disclosure, there is provided a program configured for a computer to implement functions of an analysis unit configured to analyze a hypnogram representing a time-series change of a sleep state of a subject and to calculate a sleep parameter representing a feature of sleep of the subject, and an estimation unit configured to estimate an evaluation on sleep of the subject based on a database, in which a diagnosis result of the hypnogram as sample data is registered, and the calculated sleep parameter.

According to an embodiment of the present disclosure, a hypnogram representing a time-series change of a sleep state of a subject is analyzed, and a sleep parameter representing a feature of sleep of the subject is calculated, and an evaluation on sleep of the subject based on a database is estimated, in which a diagnosis result of the hypnogram as sample data is calculated, and the obtained sleep parameter.

According to an embodiment of the present disclosure, it is possible to evaluate the quality of sleep based on a measurement result of a bio-signal during sleep.

### BRIEF DESCRIPTION OF THE DRAWINGS

FIG. 1 is a block diagram illustrating a structure of a sleep evaluation device according to an embodiment of the present disclosure;

FIG. 2 is a view illustrating a hypnogram;

FIG. 3 is a view illustrating terms related to sleep;

FIG. 4 is a view illustrating a correspondence relationship between sleep parameters and evaluation items;

FIG. 5 is a flow chart illustrating a sleep evaluation process;

FIG. 6 is a view illustrating a diagnosis result;

FIG. 7 is a view illustrating an evaluation statement of a diagnosis result; and

FIG. 8 is a view illustrating a structure of a computer.

### DETAILED DESCRIPTION OF THE EMBODIMENT(S)

Hereinafter, preferred embodiments of the present disclosure will be described in detail with reference to the appended drawings. Note that, in this specification and the appended drawings, structural elements that have substantially the same function and structure are denoted with the same reference numerals, and repeated explanation of these structural elements is omitted.

[Structure of Sleep Evaluation Device]

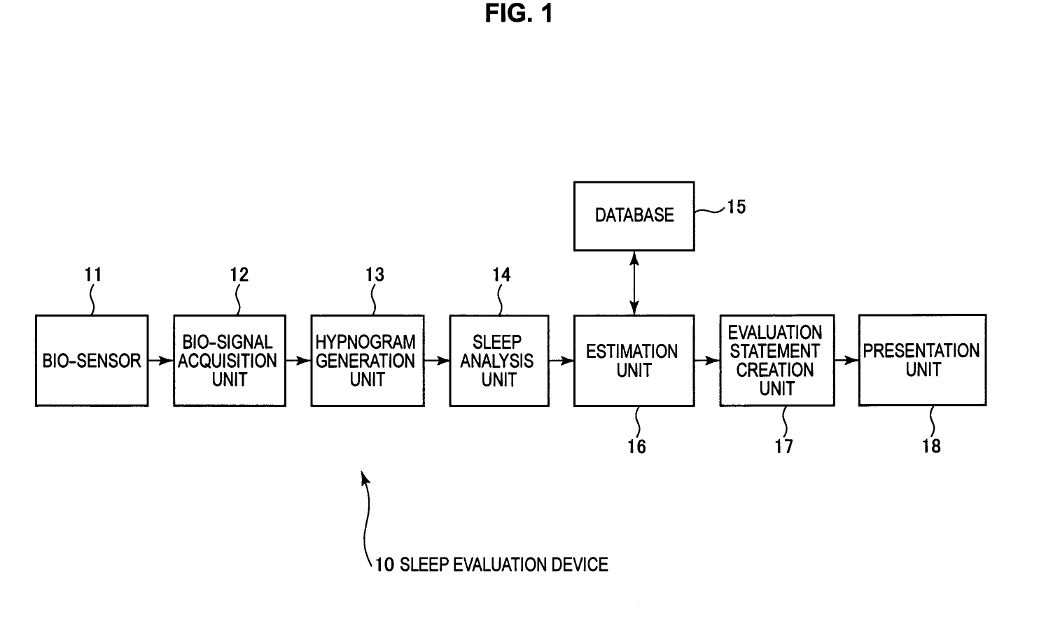

FIG. 1 is a block diagram illustrating a structure of a sleep evaluation device according to an embodiment of the present disclosure. The sleep evaluation device 10 is configured to evaluate the quality of sleep of a user (subject) based on a breathing rate, a pulse rate, a heart rate, a brain wave, a vibration due to a body movement and the like that are acquired from the user during sleep.

The sleep evaluation device 10 includes a bio-sensor 11, a bio-signal acquisition unit 12, a hypnogram generation unit 13, a sleep analysis unit 14, a database 15, an estimation unit 16, an evaluation statement creation unit 17, and a presentation unit 18.

The bio-sensor 11 is made up of a variety of sensors to measure a breathing rate, a pulse rate, a heart rate, a brain wave, a vibration due to a body movement and the like from the user during sleep. The bio-sensor 11 may store and maintain at least a measurement result of a general sleep period of time (hereinafter referred to as “bio-signal”). The bio-sensor 11 outputs the stored bio-signal to the bio-signal acquisition unit 12 at the request of the bio-signal acquisition unit 12. Further, the bio-signal 11 may output real-time measurement results to the bio-signal acquisition unit 12 in a sequential manner.

The bio-signal acquisition unit 12 acquires the bio-signal from the bio-sensor 11 and outputs the bio-signal to the hypnogram generation unit 13. The hypnogram generation unit 13 generates a hypnogram representing a time-series transition of sleep states based on the input bio-signal and outputs the hypnogram to the sleep analysis unit 14. The hypnogram will be described in detail.

The sleep analysis unit 14 calculates a plurality of sleep parameters representing features of sleep based on the generated hypnogram and outputs the sleep parameters to the estimation unit 16.

It is assumed that the database 15 contains sleep parameters and a diagnosis result of a medical specialist regarding each of a plurality of hypnograms (e.g., evaluation value A, B or C for each evaluation item and comprehensive evaluation), which have been registered beforehand, with respect to each of the plurality of hypnograms taken as samples.

The estimation unit 16 refers to the database 15 and estimates an evaluation value A, B or C for each of the evaluation items for evaluating the quality of sleep (e.g., A=100, B=50, C=0) and a probability corresponding to the evaluation value A, B or C, based on the calculated sleep parameters. Further, the estimation unit 16 calculates an expected value, which is equal to evaluation value A×probability corresponding to evaluation value A+evaluation value B×probability corresponding to evaluation value B+evaluation value C×probability corresponding to evaluation value C, based on the estimated result and outputs the expected value to the evaluation statement creation unit 17.

Specifically, for example, the estimation unit 16 employs a maximum a posteriori probability (MAP) estimation to estimate an evaluation value Y having a highest likelihood with respect to an obtained sleep parameter X based on the following equation.

Y=arg max P(Y|X)=arg max P(X|Y) P(Y)

In this equation, P(Y|X) is a posteriori probability which represents a probability of an evaluation value of Y with respect to the sleep parameter X; P(X|Y) is a likelihood which represents a distribution of X with respect to an evaluation value of Y; and P(Y) is a priori which represents a probability of an evaluation value of Y. In this case, it is assumed that the distribution of the sleep parameter X, i.e., P(X|Y), which is estimated based on a diagnosis result of a medical specialist, is registered in the database 15.

The evaluation items are estimated using kernel density estimation which is a non-parametric distribution estimation technique. Estimation of a comprehensive evaluation employs an expectation maximization (EM) algorithm using a mixed Gaussian distribution, which is a parametric distribution estimation technique.

The evaluation statement creation unit 17 creates an evaluation statement based on an expected value for the user's sleep which is input from the estimation unit 16, and outputs the evaluation statement and the expected value to the presentation unit 18. The presentation unit 18 presents the user with an expected value of each of the evaluation items for the user's sleep and the evaluation statement at the same time. The expected value of each of the evaluation items may be presented together with, for example, a relative position regarding distribution of a sample which is registered in the database 15.

[Hypnogram]

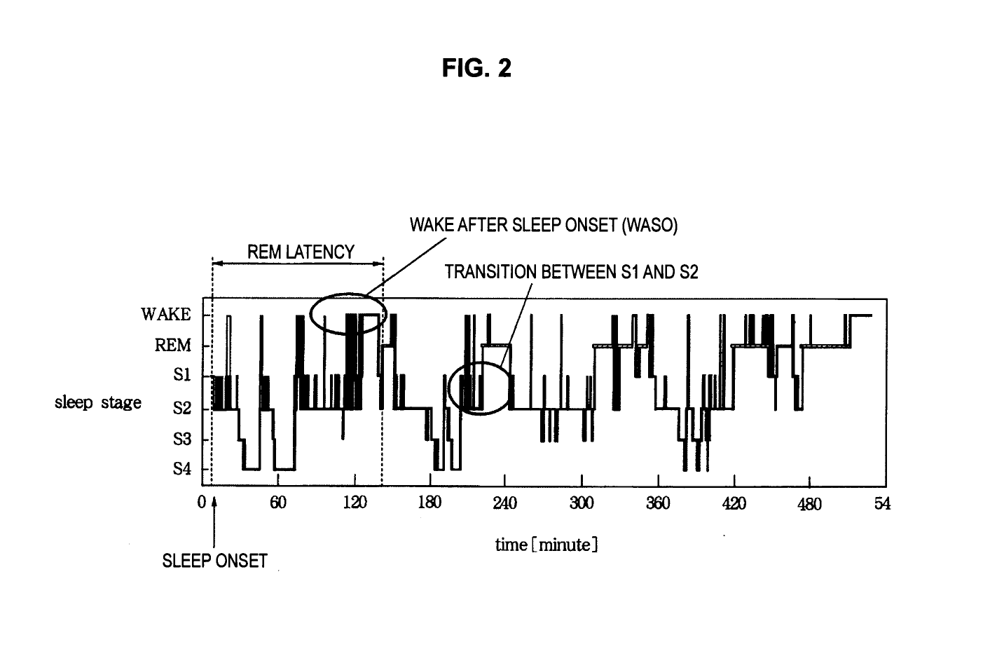

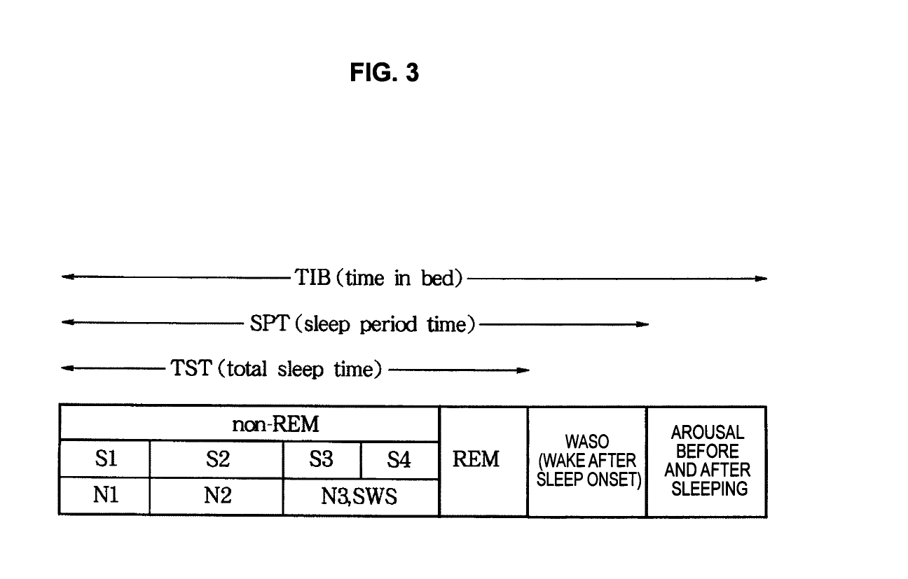

A person's sleep will be described with reference to FIGS. 2 and 3. FIG. 2 is a view illustrating a hypnogram and FIG. 3 is a view illustrating terms related to sleep.

In general, if a person goes to bed, he or she falls into a sleep state in a few minutes. Sleep is classified into two phases: rapid eye movement (REM) sleep and non-rapid eye movement (non-REM) sleep. During REM sleep, his or her physical body is asleep but his or her brain is in action. On the other hand, during non-REM sleep, his or her physical body and brain are all asleep. In general, after falling to sleep, he or she first experiences a period of non-REM sleep, and then experiences a period of REM sleep in about one or two hours. Next, he or she alternately experiences the non-REM sleep and the REM sleep.

Non-REM sleep is further classified into four stages: stage 1 (S1), stage 2 (S2), stage 3 (S3), and stage 4 (S4) in order of a lighter sleep. Specifically, the stages S3 and S4 are referred to as slow wave sleep (SWS). Alternatively, the stages S3 and S4 may be integrated into a single stage.

Terms representing sleeping time may include time in bed (TIB), sleep period time (SPT), and total sleep time (TST).

The term TIB represents a period of time between going to bed and getting up (including the arousal time before and after sleeping). The term SPT represents the time that is obtained by subtracting the arousal time before and after sleeping (i.e., a period of time between going to bed and the onset of sleep, and a period of time between waking up and getting up) from TIB. The term TST represents the time that is obtained by subtracting a period of time of wake after sleep onset (WASO) from SPT.

[Sleep Parameters and Evaluation Items]

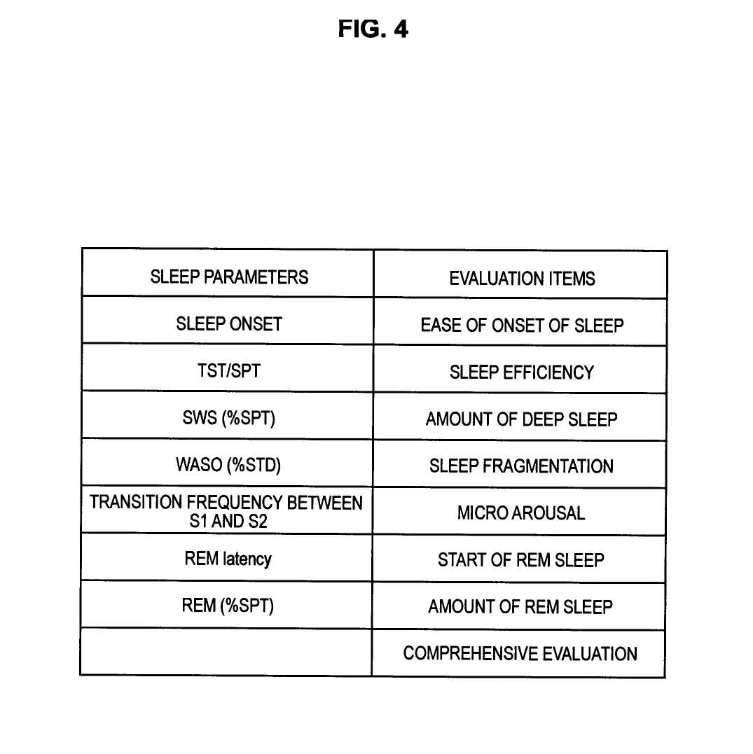

FIG. 4 is a view illustrating a correspondence relationship between sleep parameters, which are calculated from the hypnogram, and evaluation items, which are based on the sleep parameters.

Examples of the sleep parameters include sleep onset, TST/SPT, SWS(% SPT), WASO(% SPT), transition frequency between stages S1 and S2, REM latency, and REM(% SPT).

The parameter “sleep onset” represents a period of time between going to bed and the onset of sleep (where the stage S1 consecutively occurs three times or more, or the stage S2, S3, S4 or REM sleep occurs). Based on the sleep onset, the ease of the onset of sleep is evaluated.

The parameter “TST/SPT” represents the ratio of TST to SPT. Based on TST/SPT, a sleep efficiency is evaluated.

The parameter “SWS(% SPT)” represents the ratio of a period of time of the stage S3 or S4 to SPT. Based on SWS(% SPT), the amount of deep sleep is evaluated.

The parameter “WASO(% SPT)” represents the ratio of a period of time of the arousal of brain during sleep to SPE Based on WASO(% SPT), the degree of sleep fragmentation is evaluated.

From the transition frequency between the stages S1 and S2, which is one of the sleep parameters, micro arousal is evaluated.

The parameter “REM latency” represents a period of time between the onset of sleep and the appearance of the first REM sleep (sleep cycle). Based on REM latency, the start of REM sleep is evaluated.

The parameter “REM(% SPT)” represents the ratio of REM sleep time to SPT. Based on REM(% SPT), the amount of REM sleep is evaluated.

Further, based on all of the sleep parameters including sleep onset, TST/SPT, SWS(% SPT), WASO(% SPT), transition frequency of stages S1 and S2, REM latency, and REM(% SPT), the quality of sleep is comprehensively evaluated.

It may not be necessary to calculate all of the above-mentioned sleep parameters. Instead of the above-mentioned sleep parameters, other sleep parameters may be calculated.

[Description of Sleep Estimation Process]

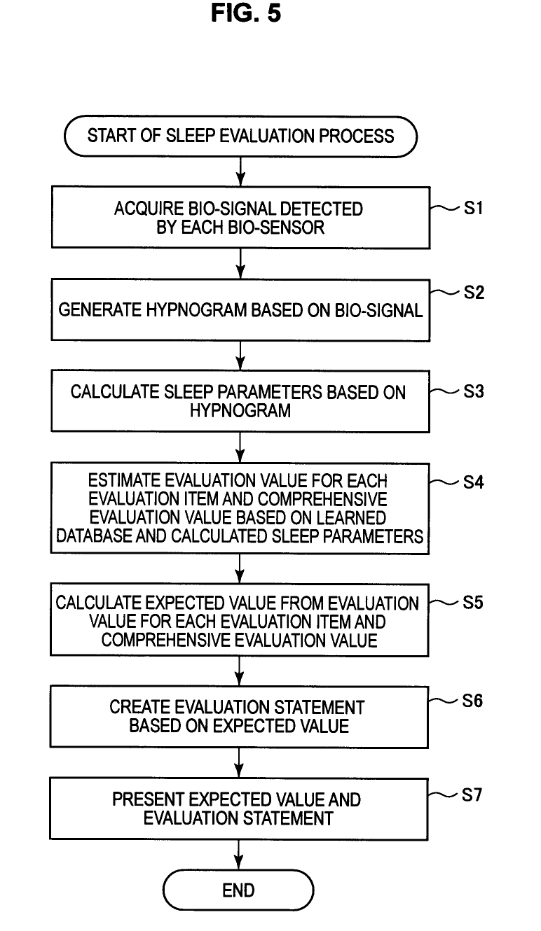

FIG. 5 is a flow chart illustrating a sleep evaluation process which is performed by the sleep evaluation device 10.

For the sleep evaluation process, it is assumed that memory built in the bio-sensor 11 stores and maintains a bio-signal detected from the user during sleep.

In step 51, the bio-signal acquisition unit 12 acquires a bio-signal from the bio-sensor 11 and outputs the bio-signal to the hypnogram generation unit 13. In step S2, the hypnogram generation unit 13 generates a hypnogram based on the bio-signal input from the bio-signal acquisition unit 12 and outputs the hypnogram to the sleep analysis unit 14.

In step S3, the sleep analysis unit 14 calculates a plurality of sleep parameters indicating features of sleep based on the generated hypnogram and outputs the sleep parameters to the estimation unit 16. In step S4, the estimation unit 16 refers to the database 15 and estimates an evaluation value A, B or C for each of evaluation items for evaluating the quality of sleep and a probability corresponding to the comprehensive evaluation value A, B or C based on the calculated sleep parameters. In step S5, the estimation unit 16 calculates an expected value from the sum of products of a setting value (e.g., 100, 50, 0) and an estimated probability for each of the evaluation values A, B and C, and outputs the expected value to the evaluation statement creation unit 17.

In step S6, the evaluation statement creation unit 17 creates an evaluation statement based on the expected value for the user's sleep, which is input from the estimation unit 16, and outputs the evaluation statement and the expected value to the presentation unit 18. In step S7, the presentation unit 18 presents the user with the expected value for each of the evaluation items for the user's sleep and the created evaluation statement.

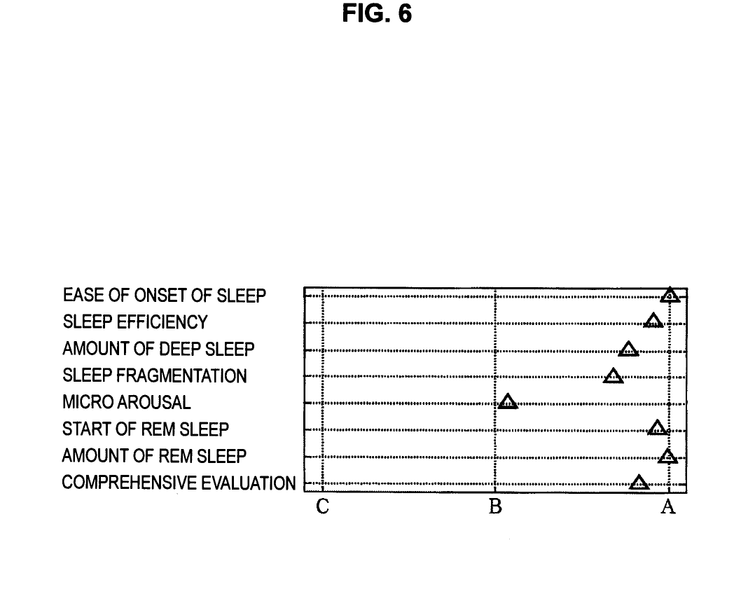

FIG. 6 is a view illustrating a screen for displaying the expected value for each of the evaluation items for the user's sleep. As shown in FIG. 6, the user may be presented with more specific evaluation values (i.e., values between the evaluation values A, B and C) using the expected values rather than the three levels of evaluation values A, B and C.

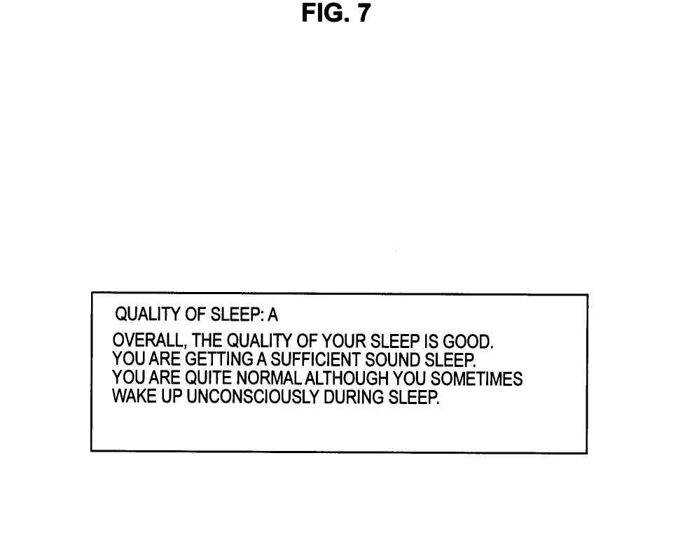

FIG. 7 is a view illustrating a screen for displaying an evaluation statement. By presenting the user with the evaluation statement, the user may understand his or her sleep more easily, compared to a case where the user is only presented with the expected value of each of the evaluation items. A detailed description of the sleep evaluation process is now completed.

### Modified Example

The elements of the sleep evaluation device 10 in FIG. 1 may be integrated into one entity or separated from each other. For example, only the bio-sensor 11 may be separated from the sleep evaluation device 10 or the presentation unit 18 may be incorporated into a terminal device, such as a portable game machine or a smart phone.

Further, for example, part of or all of the hypnogram generation unit 13, the sleep analysis unit 14, the database 15, the estimation unit 16, and the evaluation statement creation unit 17 may be installed in a server on the Internet.

A medical specialist may be allowed to access the database 15, which is installed in the server on the Internet, and additionally register his or her own diagnosis result with regard to a hypnogram, which has been registered in the database 15 and from which another medical specialist has already diagnosed. As such, the medical specialist may broaden his or her diagnosis experience.

When diagnosis results of a plurality of medical specialists for the same hypnogram are registered in the database 15, the diagnosis results may be classified and registered on a medical-specialist by medical-specialist basis since the medial specialists may have different diagnosis results from each other.

In this case, the estimation unit 16 may further include an operation input unit so that the user may select his or her desired one of the medical specialists who have registered their diagnosis results in the database 15. The estimation unit 16 may be configured to estimate an evaluation value by referring to a diagnosis result of the selected medical specialist.

The estimation unit 16 may further include an operation input unit for the user to input his or her personal opinion (self-evaluation). In this case, the estimation unit 16 may be configured to detect a registered diagnosis result, which makes it possible for the estimation unit 16 to obtain an estimated result which is similar to the user's personal opinion, from the database 15 and specify a medical specialist who has registered the detected diagnosis result. If the specified medical specialist's diagnosis result is configured to be automatically employed in estimating an evaluation value after the medical specialist has been specified, it is possible to obtain an evaluation result which is almost the same as the user's personal opinion with respect to the quality of sleep. Further, the user may be presented with information on the specified medical specialist.

The above-mentioned series of processes may be performed either by hardware or by software. If the series of processes are to be performed by software, programs of the software are installed in a computer. Examples of the computer may include a computer incorporated in a dedicated hardware, and a general-purpose personal computer that can execute a variety of functions by means of a variety of installed programs.

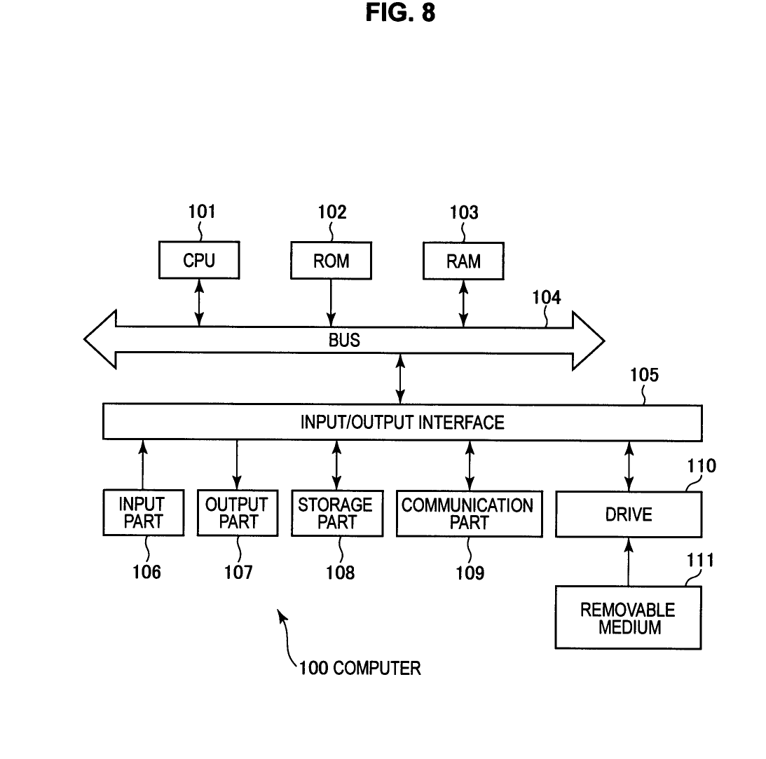

FIG. 8 is a block diagram illustrating a hardware structure of a computer that executes the series of processes by means of programs.

In the computer, a CPU (central processing unit) 101, a ROM (read-only memory) 102, and a RAM (random access memory) 103 are connected to each other through a bus 104.

An input/output interface 105 is also connected to the bus 104. The input/output interface 105 is further connected to an input unit 106, an output unit 107, a storage unit 108, a communication unit 109, and a drive 110.

The input unit 106 is made up of a keyboard, a mouse, a microphone or the like. The output unit 107 is made up of a display, a speaker or the like. The storage unit 108 is made up of a hard disc, nonvolatile memory or the like. The communication unit 109 is made up of a network interface or the like. The drive 110 drives removable media 111, such as a magnetic disc, an optical disc, a semiconductor memory or the like.

In the computer thus configured, the CPU 101 performs the above-mentioned series of processes by, for example, loading a program, which is stored in the storage unit 108, onto the RAM 103 through the input/output interface 105 and the bus 104 and executing the program.

The program executed by the computer (CPU 101) may be recorded, for example, on the removable media 111 as package media and be provided. Further, the program may be provided through wired or wireless transmission media, such as local area network, Internet, or digital satellite broadcasting.

In the computer, the program may be installed in the storage unit 108 through the input/output interface 105 by mounting the removable media 111 onto the drive 110. Further, the program may be received by the communication unit 109 through wired or wireless transmission media and installed in the storage unit 108. In addition, the program may be installed in the ROM 102 or the storage unit 108 beforehand.

It should be noted that the programs executed by the computer may be configured to be performed not only in time series in the described order in the present disclosure but also in parallel or at an appropriate timing, such as when called.

It should be understood by those skilled in the art that various modifications, combinations, sub-combinations and alterations may occur depending on design requirements and other factors insofar as they are within the scope of the appended claims or the equivalents thereof.

The present disclosure contains subject matter related to that disclosed in Japanese Priority Patent Application JP 2011-193527 filed in the Japan Patent Office on Sep. 6, 2011, the entire content of which is hereby incorporated by reference.

What is claimed is:

1. An information processing device comprising:
an analysis unit configured to analyze a hypnogram representing a time-series change of a sleep state of a subject and to calculate a sleep parameter representing a feature of sleep of the subject; and
an estimation unit configured to estimate an evaluation on sleep of the subject based on a database, in which a diagnosis result of the hypnogram as sample data is registered, and the calculated sleep parameter.

2. The information processing device according to claim 1, further comprising
a presentation unit configured to present the estimated evaluation on the sleep of the subject.

3. The information processing device according to claim 2, further comprising
a creation unit configured to create an evaluation statement based on the estimated evaluation on the sleep of the subject,
wherein the presentation unit further presents the evaluation statement.

4. The information processing device according to claim 2,
wherein the database registers one or more results obtained by diagnosis performed by one or more medical specialists with respect to the hypnogram as sample data.

5. The information processing device according to claim 4,
wherein the estimation unit includes an input unit to input a personal opinion on sleep of the subject and is configured to specify, out of the one or more medical specialists having registered diagnosis results in the database, a medical specialist, an evaluation value estimated based on a result obtained by diagnosis performed by the specified medical specialist being similar to the input personal opinion of the subject.

6. The information processing device according to claim 5,
wherein the estimation unit is configured to estimate an evaluation on sleep of the subject based on the calculated sleep parameter and the result obtained by diagnosis performed by the specified medical specialist among diagnosis results registered in the database.

7. The information processing device according to claim 2,
wherein the analysis unit is configured to calculate, as the sleep parameter, at least one of sleep onset, TST/SPT, SWS(% SPT), WASO(% SPT), transition frequency between stages S1 and S2, REM latency, or REM(% SPT).

8. The information processing device according to claim 2, further comprising:
an acquisition unit configured to acquire a bio-signal measured from a subject during sleep; and
a generation unit configured to generate the hypnogram based on the acquired bio-signal.

9. The information processing device according to claim 8, further comprising:
a bio-sensor configured to measure the bio-signal from a subject during sleep.

10. An information processing method of an information processing device, the information processing method performed by the information processing device, comprising:
analyzing a hypnogram representing a time-series change of a sleep state of a subject and calculating a sleep parameter representing a feature of sleep of the subject; and
estimating an evaluation on sleep of the subject based on a database, in which a diagnosis result of the hypnogram as sample data is registered, and the calculated sleep parameter.

11. A program configured for a computer to implement functions of:
an analysis unit configured to analyze a hypnogram representing a time-series change of a sleep state of a subject and to calculate a sleep parameter representing a feature of sleep of the subject; and
an estimation unit configured to estimate an evaluation on sleep of the subject based on a database, in which a diagnosis result of the hypnogram as sample data is registered, and the calculated sleep parameter.
